(19) United States
(12) Patent Application Publication
(10) Pub. No.: US 2024/0055076 A1
(43) Pub. Date: Feb. 15, 2024

(54) TREATMENT OF DISEASES ASSOCIATED WITH VARIANT NOVEL OPEN READING FRAMES

(71) Applicants: Cambridge Enterprise Limited, Cambridge (GB); International Centre for Genetic Engineering and Biotechnology, Delhi (IN)

(72) Inventor: Sudhakaran PRABAKARAN, Acton, MA (US)

(21) Appl. No.: 18/266,387

(22) PCT Filed: Dec. 9, 2021
(86) PCT No.: PCT/IB2021/061475

§ 371 Date: Jun. 9, 2023

Related U.S. Application Data
(60) Provisional application No. 63/123,454, filed on Dec. 9, 2020.

(51) Int. Cl. G16B 35/00; G16B 20/20; C12N 15/10

(52) U.S. Cl. CPC G16B 35/00; G16B 20/20; C12N 15/1089

(57) ABSTRACT

The present application features methods of treating a disease associated with a genetic variant. The genetic variant is also present within a novel open reading frame (nORF) associated with the gene in which the variant encodes either the gain or loss of a stop codon.

### CROSS-REFERENCE TO RELATED APPLICATIONS

This application claims the benefit of U.S. Provisional Application No. 63/123,454 filed on Dec. 9, 2020, which is incorporated herein by reference in its entirety.

### BACKGROUND OF THE INVENTION

Many diseases are caused by genetic mutations that present, at face, to be benign to the gene or the canonical protein encoded by the gene that is associated with the disease. Thus, it is unclear how certain benign genetic variants contribute to disease pathology under these circumstances. Furthermore, as it is unclear how these mutations contribute to the underlying disease pathology, providing an effective therapeutic remains a challenging endeavor. Accordingly, new methods of diagnosis and treatment are needed to better understand how these benign variants cause disease across a wide range of conditions.

### SUMMARY OF THE INVENTION

In one aspect, the invention features a method of treating a disease in a subject by identifying a sequence variant in a gene including a canonical open reading frame (cORF) and a disease associated therewith. The method includes identifying a sequence of a novel open reading frame (nORF) of the gene that is distinct from the cORF, wherein the nORF is present in (i) an overlapping region of the cORF in an alternate reading frame, (ii) a 5′ untranslated region (UTR) of the cORF, (iii) a 3′ UTR of the cORF, (iv) an intronic region of the cORF, or (v) an intergenic region of the cORF, wherein the sequence variant encodes the loss of a stop codon or portion thereof in the nORF, and wherein the absence of the sequence variant does not encode the loss of the stop codon or portion thereof in the nORF; and administering an inhibitor of the protein encoded by the nORF to the subject treat the disease.

In another aspect, the invention features a method of treating a disease in a subject by administering an inhibitor of a protein encoded by a nORF containing a stop codon to the subject. The subject may have previously been identified with a sequence variant in a gene including a cORF associated with the disease; and a sequence of the nORF of the gene that is distinct from the cORF, wherein the nORF is present in (i) an overlapping region of the cORF in an alternate reading frame, (ii) a 5′ untranslated region (UTR) of the cORF, (iii) a 3′ UTR of the cORF, (iv) an intronic region of the cORF, or (v) an intergenic region of the cORF, wherein the sequence variant encodes the loss of a stop codon in the nORF, and wherein the absence of the sequence variant does not encode the loss of the stop codon in the nORF.

In another aspect, the invention features a method of treating a disease in a subject by identifying a sequence variant in a gene including a cORF and a disease associated therewith. The method includes identifying a sequence of a nORF of the gene that is distinct from the cORF, wherein the nORF is present in (i) an overlapping region of the cORF in an alternate reading frame, (ii) a 5′ untranslated region (UTR) of the cORF, (iii) a 3′ UTR of the cORF, (iv) an intronic region of the cORF, or (v) an intergenic region of the cORF, wherein the sequence variant encodes a stop codon or portion thereof in the nORF, and wherein the absence of the sequence variant does not encode the stop codon or portion thereof in the nORF; and administering an inhibitor of the protein encoded by the nORF to the subject treat the disease.

In another aspect, the invention features a method of treating a disease in a subject by administering an inhibitor of a protein encoded by a nORF to the subject. The subject may have been previously been identified with a sequence variant in a gene including a cORF associated with the disease; and a sequence of the nORF of the gene that is distinct from the cORF, wherein the nORF is present in (i) an overlapping region of the cORF in an alternate reading frame, (ii) a 5′ untranslated region (UTR) of the cORF, (iii) a 3′ UTR of the cORF, (iv) an intronic region of the cORF, or (v) an intergenic region of the cORF, wherein the sequence variant encodes a stop codon in the nORF, and wherein the absence of the sequence variant does not encode the stop codon in the nORF.

In some embodiments of any of the above aspects, the nORF is present in an overlapping region of the cORF in an alternate reading frame.

In some embodiments of any of the above aspects, the inhibitor is a small molecule, a polynucleotide, or a polypeptide. The polynucleotide may be, e.g., a miRNA, an antisense RNA, an shRNA, or an siRNA. The polypeptide may be, e.g., an antibody or antigen-binding fragment thereof (e.g., an scFv).

In some embodiments, the inhibitor is encoded by a vector, such as a viral vector. The viral vector may be selected, for example, from the group consisting of a Retroviridae family virus, an adenovirus, a parvovirus, a coronavirus, a rhabdovirus, a paramyxovirus, a picornavirus, an alphavirus, a herpes virus, and a poxvirus. The parvovirus viral vector may be, for example, an adeno-associated virus (AAV) vector.

In some embodiments, the viral vector is a Retroviridae family viral vector (e.g., a lentiviral vector, an alpharetroviral vector, or a gammaretroviral vector). The Retroviridae family viral vector may include one or more of the following: a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

In some embodiments, the viral vector is a pseudotyped viral vector. The pseudotyped viral vector may be selected, for example, from the group consisting of a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus. The pseudotyped viral vector may be, e.g., a lentiviral vector.

In some embodiments, the pseudotyped viral vector includes one or more envelope proteins from a virus selected from vesicular stomatitis virus (VSV), RD114 virus, murine leukemia virus (MLV), feline leukemia virus (FeLV), Venezuelan equine encephalitis virus (VEE), human foamy virus (HFV), walleye dermal sarcoma virus (WDSV), Semliki Forest virus (SFV), Rabies virus, avian leukosis virus (ALV), bovine immunodeficiency virus (BIV), bovine leukemia virus (BLV), Epstein-Barr virus (EBV), Caprine arthritis encephalitis virus (CAEV), Sin Nombre virus (SNV), Cherry Twisted Leaf virus (ChTLV), Simian T-cell leukemia virus (STLV), Mason-Pfizer monkey virus (MPMV), squirrel monkey retrovirus (SMRV), Rous-associated virus (RAV), Fujinami sarcoma virus (FuSV), avian carcinoma virus (MH2), avian encephalomyelitis virus (AEV), Alfa mosaic virus (AMV), avian sarcoma virus CT10, and equine infectious anemia virus (EIAV).

In some embodiments, the pseudotyped viral vector includes a VSV-G envelope protein.

In another aspect, the invention features a method of treating a disease in a subject by identifying a sequence variant in a gene including a cORF and a disease associated therewith. The method includes the step of identifying a sequence of a nORF of the gene that is distinct from the cORF, wherein the nORF is present in (i) an overlapping region of the cORF in an alternate reading frame, (ii) a 5′ untranslated region (UTR) of the cORF, (iii) a 3′ UTR of the cORF, (iv) an intronic region of the cORF, or (v) an intergenic region of the cORF, wherein the sequence variant encodes the loss of a stop codon or portion thereof in the nORF, and wherein the absence of the sequence variant does not encode the loss of the stop codon or portion thereof in the nORF; and administering a protein encoded by the wild-type (WT) nORF containing the stop codon to the subject treat the disease.

In another aspect, the invention features a method of treating a disease in a subject by administering a protein encoded by a WT nORF containing a stop codon to the subject. The subject may have previously been identified with a sequence variant in a gene including a cORF associated with the disease; and a sequence of the nORF of the gene that is distinct from the cORF, wherein the nORF is present in (i) an overlapping region of the cORF in an alternate reading frame, (ii) a 5′ untranslated region (UTR) of the cORF, (iii) a 3′ UTR of the cORF, (iv) an intronic region of the cORF, or (v) an intergenic region of the cORF, wherein the sequence variant encodes the loss of a stop codon in the nORF, and wherein the absence of the sequence variant does not encode the loss of the stop codon in the nORF.

In another aspect, the invention features a method of treating a disease in a subject by identifying a sequence variant in a gene including a cORF and a disease associated therewith. The method includes identifying a sequence of a nORF of the gene that is distinct from the cORF, wherein the nORF is present in (i) an overlapping region of the cORF in an alternate reading frame, (ii) a 5′ untranslated region (UTR) of the cORF, (iii) a 3′ UTR of the cORF, (iv) an intronic region of the cORF, or (v) an intergenic region of the cORF, wherein the sequence variant encodes a stop codon or portion thereof in the nORF, and wherein the absence of the sequence variant does not encode the variant stop codon or portion thereof in the nORF; and administering a protein encoded by the WT nORF without the stop codon to the subject treat the disease.

In another aspect, the invention features a method of treating a disease in a subject including administering a protein encoded by a WT nORF to the subject. The subject may have previously been identified with a sequence variant in a gene including a cORF associated with the disease; and a sequence of the nORF of the gene that is distinct from the cORF, wherein the nORF is present in (i) an overlapping region of the cORF in an alternate reading frame, (ii) a 5′ untranslated region (UTR) of the cORF, (iii) a 3′ UTR of the cORF, (iv) an intronic region of the cORF, or (v) an intergenic region of the cORF, wherein the sequence variant encodes the stop codon in the nORF, and wherein the absence of the sequence variant in the WT nORF does not encode the stop codon in the nORF.

In some embodiments of any of the above aspects, the nORF is present in an overlapping region of the cORF in an alternate reading frame.

In some embodiments of any of the above aspects, the method includes restoring the encoded protein product of the WT nORF without the sequence variant. The method may include providing the protein product or a polynucleotide encoding the protein product.

In some embodiments of any of the above aspects, the method includes the step of providing a vector including the polynucleotide encoding the protein product.

In some embodiments, the vector is a viral vector. The viral vector may be selected, for example, from the group consisting of a Retroviridae family virus, an adenovirus, a parvovirus, a coronavirus, a rhabdovirus, a paramyxovirus, a picornavirus, an alphavirus, a herpes virus, and a poxvirus. The parvovirus viral vector may be an adeno-associated virus (AAV) vector.

In some embodiments, the viral vector is a Retroviridae family viral vector (e.g., a lentiviral vector, an alpharetroviral vector, or a gammaretroviral vector). The Retroviridae family viral vector may include one or more of the following: a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

In some embodiments, the viral vector is a pseudotyped viral vector. The pseudotyped viral vector may be selected, for example, from the group consisting of a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus. The pseudotyped viral vector may be a lentiviral vector.

In some embodiments, the pseudotyped viral vector includes one or more envelope proteins from a virus selected from VSV, RD114 virus, MLV, FeLV, VEE, HFV, WDSV, SFV, Rabies virus, ALV, BIV, BLV, EBV, CAEV, SNV, ChTLV, STLV, MPMV, SMRV, RAV, FuSV, MH2, AEV, AMV, avian sarcoma virus CT10, and EIAV.

In some embodiments, the pseudotyped viral vector includes a VSV-G envelope protein.

In some embodiments of any of the above aspects, the encoded protein product of the nORF is less than about 100 amino acids.

In some embodiments, the method further includes performing a statistical analysis between the variant in the nORF and the disease. The statistical analysis may measure a positive or negative association between the variant in the nORF and the disease.

In some embodiments, the disease is cancer (e.g., breast cancer or Medullary thyroid carcinoma). The gene may be BRCA2. The gene may be RET. In some embodiments, the gene is selected from the group consisting of TTN, TP53, EGFR, FAT1, MACF1, TSC2, NOTCH1, ANK2, MYC, NEB, NLRP2, CREBBP, ANAPC5, DST, EXT1, NF1, AR1D1A, ATM, CTNNA2, and JAK1.

When treating cancer, the method may reduce the size (e.g., by 10%, 20%, 30%, 40%, 50%, 60%, 70%, 80%, 90%, 95%, or 99%) of a tumor (e.g., a breast tumor).

In some embodiments (a) the disease is Leber congenital amaurosis, and the gene is NMNAT1; (b) the disease is Charcot Marie Tooth disease type 1B, and the gene is MPZ; (c) the disease is Spastic paraplegia autosomal dominant, and the gene is SPAST; (d) the disease is Pulmonary arterial hypertension, and the gene is BMPR2; (e) the disease is Coproporphyria, and the gene is CPOX; (f) the disease is Epileptic encephalopathy early onset, and the gene is ALDH7A1; (g) the disease is Alpha-AASA dehydrogenase deficiency, and the gene is ALDH7A1; (h) the disease is Mucopolysaccharidosis VII, and the gene is GUSB; (i) the disease is Cowden disease, and the gene is PTEN; (j) the disease is Beta thalassaemia, and the gene is HBB; (k) the disease is Multiple endocrine neoplasia 1, and the gene is MEN1; (l) the disease is Cerebellar ataxia recurrent liver failure peripheral neuropathy and short stature, and the gene is SCYL1; (m) the disease is Pituitary adenoma, and the gene is AIP; (n) the disease is Marfan syndrome, and the gene is FBN1; (o) the disease is Gangliosidosis GM2, and the gene is HEXA; (p) the disease is Leigh syndrome, and the gene is MRPS34; (q) the disease is Apparent mineralocorticoid excess, and the gene is HSD11B2; (r) the disease is Neurofibromatosis 1, and the gene is NF1; (s) the disease is Osteogenesis imperfecta 1, and the gene is COL1A1; (t) the disease is Hypercholesterolaemia, and the gene is LDLR; (u) the disease is Aicardi-Goutlbres syndrome, and the gene is RNASEH2A; (v) the disease is Hyperferritinaemia cataract syndrome, and the gene is FTL; (w) the disease is Retinitis pigmentosa, and the gene is PRPF31; (x) the disease is Neurofibromatosis 2, and the gene is NF2; (y) the disease is Pyridoxine-dependent epilepsy, and the gene is ALDH7A1; (z) the disease is Hypotrichosis 4, and the gene is HR; (aa) the disease is Somatotroph adenoma, and the gene is AIP; (bb) the disease is Gm2 gangliosidosis, subacute, and the gene is HEXA; (cc) the disease is Combined oxidative phosphorylation deficiency 32, and the gene is MRPS34; or (dd) the disease is Alcardi Goutieres syndrome 4, and the gene is MRPS34.

In some embodiments, the disease and the gene are selected from Table 3.

In some embodiments, the disease and the gene are selected from Table 4.

In some embodiments, the disease and the gene are selected from Table 5.

In some embodiments, the disease is selected from the list consisting of amyotrophic lateral sclerosis, martan syndrome, myasthenic syndrome, congenital, Charcot-Marle-Tooth disease, neural tube defects, Ehlers-Danlos syndrome, cortical cataract, dyssegmental dysplasia, Diamond-Blackfan anemia, familial hypercholesterolemia, reticular dysgenesis, dystonia, severe congenital neutropenia, hyperinsulinism, noonan syndrome, mitochondrial cytopathy, Melnick-Needles syndrome, frontometaphyseal dysplasia, spastic paraplegia, Baraitser-Winter syndrome, peripheral axonal neuropathy, mucopolysaccharidosis, lissencephaly 2, maple syrup urine disease, myofibrillar myopathy, Pitt-Hopkins-like syndrome 1, weaver syndrome, arrhythmia, cardiomyopathy, glycogen storage disease of heart, neuronal ceroid lipofuscinosis, primary autosomal recessive microcephaly 1, Werner syndrome, Spherocytosis, Waardenburg syndrome, ciliary dyskinesia, epidermolysis bullosa simplex, Brown-Vialetto-Van Laere syndrome, amyotrophic lateral sclerosis, hyperphosphatasia with mental retardation syndrome, distal arthrogryposis, choreoacanthocytosis, phosphoserine aminotransferase deficiency, spinal muscular atrophy, congenital cataract, thoracic aortic aneurysm and aortic dissection, familial dysautonomia, Bardet-Biedl syndrome, amyloidosis, early infantile epileptic encephalopathy, Osler hemorrhagic telangiectasia syndrome, coenzyme Q10 deficiency, Walker-Warburg congenital muscular dystrophy, spinocerebellar ataxia autosomal recessive, Leigh syndrome, Ehlers-Danlos syndrome, Adams-Oliver syndrome, congenital generalized lipodystrophy, Barakat syndrome, primary open angle glaucoma, Warburg micro syndrome, long QT syndrome, multiple endocrine neoplasia, pol III-related leukodystrophy, moyamoya disease|, dilated cardiomyopathy, cutis laxa-corneal clouding-oligophrenla syndrome, Infantile spasms, Hermansky-Pudlak syndrome, Medulloblastoma, myofibrillar myopathy, Costello syndrome, seizure, neuronal ceroid lipofuscinosis, Beckwith-Wiedemann syndrome, Stormorken syndrome, neuronal ceroid lipotuscinosis, Sveinsson chorloretinal atrophy, Wilms tumor, peroxisome biogenesis disorder, syndactyly Cenani Lenz type, xeroderma pigmentosum, hereditary paraganglioma-pheochromocytoma syndromes, multiple endocrine neoplasia, type 1, autosomal recessive cutis laxa type 1, osteopetrosis autosomal recessive 1, osteogenesis imperfecta, recessive, Papillon-Lefevre syndrome, ataxia-telangiectasia syndrome, myofibrillar myopathy, 6-pyruvoyl-tetrahydropterin synthase deficiency, glycogen storage disease, type I, glucose-6-phosphate transport defect, pseudohypoaldosteronism type 2C, pseudohypoaldosteronism type 1, epidermolysis bullosa simplex, keratosis follicularls, Troyer syndrome, neuronal ceroid lipofuscinosis, nemallne myopathy 7, elliptocytosis, methylmalonate semialdehyde dehydrogenase deficiency, ventricular tachycardia, catecholaminergic polymorphic, herpes simplex encephalitis, mosaic variegated aneuploidy, arginine:glycine amidinotransferase deficiency, marfan syndrome, ectopla lentis, Griscelli syndrome type 2, fanconi anemia, progressive sclerosing poliodystrophy, Bloom syndrome, Weill-Marchesani-like syndrome, bare lymphocyte syndrome 2, EEM syndrome, Li-Fraumeni syndrome, Meler-Gorlin syndrome, naxos disease, osteogenesis imperfecta, carney complex, type 1, Howel-Evans syndrome, Majeed syndrome, Niemann-Pick disease, type C. Peutz-Jeghers syndrome, lipodystrophy, partial, acquired, leprechaunism syndrome, rhabdoid tumor predisposition syndrome 2, Alcardi Goutieres syndrome 4, retinitis pigmentosa, recessive, alagille syndrome 1, dyskeratosis congenita, pseudoinflammatory fundus dystrophy, adenylosuccinate lyase deficiency, duchenne muscular dystrophy, Wilson-Turner X-linked mental retardation syndrome, Melnick-Needles syndrome, transcobalamin 11 deficiency, nephronophthisis-like nephropathy, and Borjeson-Forssman-Lehmann syndrome.

### Definitions

As used herein, a “novel open reading frame” or “nORF” refers to an open reading frame that is transcribed in a cell and consists of a sequence that is present in a gene but is distinct from a canonical open reading frame transcribed from the gene. The nORF may be present in (i) an overlapping region of the cORF in an alternate reading frame, (ii) a 5′ untranslated region (UTR) of the cORF, (ill) a 3′ UTR of the cORF, (iv) an intronic region of the cORF, or (v) an intergenic region of the cORF.

As used herein, a “canonical open reading frame” or “cORF” refers to an open reading frame that is transcribed in a cell and its associated genetic elements, including the 5′ UTR, the 3′ UTR, the intronic regions, the exonic regions, and the intergenic regions flanking the gene that includes the cORF. A cORF includes either the primary open reading frame that is expressed from a gene, the most abundantly expressed open reading frame expressed from a gene, or an ORF that is annotated in a publicly available database as the primary and/or most abundantly expressed open reading frame from a gene.

### BRIEF DESCRIPTION OF THE DRAWINGS

FIG. 1 is a schematic drawing showing an example of removing in-frame entries where two small ORFs overlap the CDS of the RICA gene. The ORF in the same frame as the RICA CDS is removed from the dataset as indicated by the cross, whereas the second ORF in a different frame is retained in the dataset, indicated by the arrow.

FIGS. 2A-2E are schematic drawings reinterpreting COSMIC, HGMD and ClinVar mutations in the context of nORFs. The canonical consequence and nORF consequence of (FIG. 2A) 109K somatic cancer mutations from COSMIC, (FIG. 2B) 1.8K disease mutations from HGMD, and (FIG. 2C) 5.3K disease mutations from ClinVar. Bins with 10 or fewer variants not shown. These mutations would likely be interpreted as benign or missense in canonical genes but may have more severe consequences in nORFs. (FIG. 2D) A theoretical example of a disease variant that results in a synonymous mutation in canonical CDS but (FIG. 2E) a stop gain mutation in a nORF from an alternative reading frame.

### DETAILED DESCRIPTION

Described herein are methods of diagnosing and treating a disease associated with a genetic variant. Many diseases are caused by a seemingly benign mutation in a gene that is associated with the disease. However, it was previously unclear how certain benign genetic variants contribute to disease pathology. The present invention is premised, in part, upon the discovery that certain genetic variants are also present in a novel open reading frame (nORF) that is distinct from the canonical open reading frame (cORF) of the gene. In these instances, the genetic variant imparts a deleterious effect on the nORF, with or without substantially impacting the protein encoded by the cORF. In particular, the present invention features methods of treating diseases associated with a variant nORF in which the mutation encodes the gain or loss of a stop codon (i.e., stop-gain or stop-loss, respectively) in the nORF. When the mutation encodes the gain or loss of a stop codon, the gene product encoded by the variant nORF is either shorter or longer than the WT nORF. However, the variant may have no substantial effect on the cORF as the mutation may be conservative or silent to the protein encoded by the cORF. The methods of diagnosis and treatment are described in more detail below.

### Methods of Diagnosis

Genetic testing offers one avenue by which a patient may be diagnosed as having or is at risk of developing a particular disease. For example, a genetic analysis can be used to determine whether a patient has a mutation in an endogenous gene associated with a disease. The mutation may be present in any region of the gene, such as within the cORF, a 5′ untranslated region (UTR) of the cORF, a 3′ UTR of the cORF, an intronic region of the cORF, or an intergenic region of the cORF. The mutation is also present in an nORF. The nORF may be present within an overlapping region of the cORF in an alternate reading frame, a 5′ untranslated region (UTR) of the cORF, a 3′ UTR of the cORF, an intronic region of the cORF, or an intergenic region of the cORF. In some embodiments, the nORF is present in an overlapping region of the cORF in an alternate reading frame.

Exemplary genetic tests that can be used to determine whether a patient has such a mutation in the gene or the nORF include polymerase chain reaction (PCR) methods known in the art, such as DNA and RNA sequencing. In some embodiments, the subject is identified as having a certain mutation in a gene, and this mutation may be annotated in a publicly available database as being associated with a certain disease. nORF sequences may be identified de novo, e.g., using computational or statistical methods. Furthermore, nORF sequences may be identified from publicly available databases in genomic sequences in which the nORF was not previously identified and/or annotated as a sequence that was expressed and/or translated.

nORF sequences may be identified as being linked to a particular disease by using a statistical analysis between the variant in the nORF and the disease. The statistical analysis may measure a positive or negative association between the variant in the nORF and the disease (see, e.g., Example 1).

To examine the functional importance of a nORF separately from a canonical coding sequence, variant frequencies from datasets, such as the Genome Aggregation Database, may be used.

### Methods of Treatment

The invention features methods of treating a subject having a disease associated variant in an nORF that encodes a loss or gain of a stop codon. The subject may be first determined to have the stop-gain or stop-loss variant and then may be subsequently be treated for the disease. The subject may have previously been determined to have the stop-gain or stop-loss variant and is then treated for the disease. The treatment varies according to the variant nORF associated with the disease. For example, the treatment may include an inhibitor that targets the variant nORF (e.g., stop-gain or stop-loss variant). Alternatively, or in addition, the treatment may include providing the WT nORF or a protein encoded by the WT nORF without the sequence variant.

### Inhibitors

The methods of treatment and diagnosis described herein may include providing an inhibitor that targets the variant nORF. The inhibitor may reduce an amount or activity of the variant nORF, such as to prevent the deleterious effect of the variant nORF. The inhibitor may target the polynucleotide containing the nORF or the protein encoded by the nORF. The inhibitor may be, e.g., a small molecule, a polynucleotide, or a polypeptide. Suitable small molecules may be determined or identified by using computational analysis based on the structure of the variant nORF as determined by a protein folding algorithm. The small molecule may target any region of the variant nORF. The small molecule may target the nORF or the protein encoded by the nORF. Suitable polypeptides for reducing an activity or amount of the variant nORF include, for example, an antibody or antigen-binding fragment thereof that binds to the variant nORF (e.g., a single chain antibody or antigen-binding fragment thereof). Suitable polynucleotides that can reduce an amount or activity of the variant nORF include RNA. For example, an RNA for reducing an activity or amount of the variant nORF may be, for example, a miRNA, an antisense RNA, an shRNA, or an siRNA. The miRNA, antisense RNA, shRNA, or siRNA may target a region of RNA (e.g., variant nORF gene) to reduce expression of the variant nORF. The polynucleotide may be an aptamer, e.g., an RNA aptamer that binds to and/or reduces an amount and/or activity of the variant nORF or the protein encoded by the variant nORF. The inhibitor may be provided directly or may be provided by a vector (e.g., a viral vector) encoding the inhibitor. The inhibitor may be formulated, e.g., in a pharmaceutical composition containing a pharmaceutically acceptable carrier. The composition can be administered by any suitable method known in the art to the skilled artisan. The composition (e.g., a vector, e.g., a viral vector) may be formulated in a virus or a virus-like particle.

Nucleic Acid Mediated Knockdown

Using the compositions and methods described herein, a patient with a disease may be administered an interfering RNA molecule, a composition containing the same, or a vector encoding the same, so as to suppress the expression of a variant nORF. Exemplary interfering RNA molecules that may be used in conjunction with the compositions and methods described herein are siRNA molecules, miRNA molecules, shRNA molecules, and antisense RNA molecules, among others. In the case of siRNA molecules, the siRNA may be single stranded or double stranded. miRNA molecules, in contrast, are single-stranded molecules that form a hairpin, thereby adopting a hydrogen-bonded structure reminiscent of a nucleic acid duplex. In either case, the interfering RNA may contain an antisense or “guide” strand that anneals (e.g., by way of complementarity) to the repeat-expanded mutant RNA target. The interfering RNA may also contain a “passenger” strand that is complementary to the guide strand and, thus, may have the same nucleic acid sequence as the RNA target.

siRNA is a class of short (e.g., 20-25 nt) double-stranded non-coding RNA that operates within the RNA interference pathway. siRNA may interfere with expression of the variant nORF gene with complementary nucleotide sequences by degrading mRNA (via the Dicer and RISC pathways) after transcription, thereby preventing translation. miRNA is another short (e.g., about 22 nucleotides) non-coding RNA molecule that functions in RNA silencing and post-transcriptional regulation of gene expression. miRNAs function via base-pairing with complementary sequences within mRNA molecules, thereby leading to cleavage of the mRNA strand into two pieces and destabilization of the mRNA through shortening of its poly(A) tail. shRNA is an artificial RNA molecule with a tight hairpin turn that can be used to silence target gene expression via RNA interference. Antisense RNA are also short single stranded molecules that hybridize to a target RNA and prevent translation by occluding the translation machinery, thereby reducing expression of the target (e.g., the variant nORF).

Antibody Mediated Knockdown

Using the compositions and methods described herein, a patient with a disease may be provided an antibody or antigen-binding fragment thereof, a composition containing the same, a vector encoding the same, or a composition of cells containing a vector encoding the same, so as to suppress or reduce the activity of the variant nORF. In some embodiments of the compositions and methods described herein, an antibody or antigen-biding fragment thereof may be used that binds to and reduces or eliminates the activity of the variant nORF. The antibody may be monoclonal or polyclonal. In some embodiments, the antigen-binding fragment is an antibody that lacks the Fc portion, an F(ab′)2, a Fab, an Fv, or an scFv. The antigen-binding fragment may be an scFv.

One of ordinary skill in the art will appreciate that an antibody may include four polypeptides: two identical copies of a heavy chain polypeptide and two copies of a light chain polypeptide. Each of the heavy chains contains one N-terminal variable (V_{H}) region and three C-terminal constant (CH1, CH2 and CH3) regions, and each light chain contains one N-terminal variable (V_{L}) region and one C-terminal constant (C_{L}) region. Thus, one of skill in the art would appreciate that as described herein, a vector that includes a transgene that encodes a polypeptide that is an antibody may be, e.g., a single transgene that encodes a plurality of polypeptides. Also contemplated is a vector that includes a plurality of transgenes, each transgene encoding a separate polypeptide of the antibody. All variations are contemplated herein. The variable regions of each pair of light and heavy chains form the antigen binding site of an antibody. The transgene which encodes an antibody directed against the variant nORF can include one or more transgene sequences, each of which encodes one or more of the heavy and/or light chain polypeptides of an antibody. In this respect, the transgene sequence which encodes an antibody directed against the variant nORF can include a single transgene sequence that encodes the two heavy chain polypeptides and the two light chain polypeptides of an antibody. Alternatively, the transgene sequence which encodes an antibody directed against the variant nORF can include a first transgene sequence that encodes both heavy chain polypeptides of an antibody, and a second transgene sequence that encodes both light chain polypeptides of an antibody. In yet another embodiment, the transgene sequence which encodes an antibody can include a first transgene sequence encoding a first heavy chain polypeptide of an antibody, a second transgene sequence encoding a second heavy chain polypeptide of an antibody, a third transgene sequence encoding a first light chain polypeptide of an antibody, and a fourth transgene sequence encoding a second light chain polypeptide of an antibody.

In some embodiments, the transgene that encodes the antibody includes a single open reading frame encoding a heavy chain and a light chain, and each chain is separated by a protease cleavage site.

In some embodiments, the transgene encodes a single open reading frame encoding both heavy chains and both light chains, and each chain is separate by protease cleavage site.

In some embodiments, full-length antibody expression can be achieved from a single transgene cassette using 2A peptides, such as foot-and-mouth disease virus (FMDV) equine rhinitis A, porcine teschovirus-1, and Thosea asigna virus 2A peptides, which are used to link two or more genes and allow the translated polypeptide to be self-cleaved into individual polypeptide chains (e.g., heavy chain and light chain, or two heavy chains and two light chains). Thus, in some embodiments, the transgene encodes a 2A peptide in between the heavy and light chains, optionally with a flexible linker flanking the 2A peptide (e.g., GSG linker). The transgene may further include one or more engineered cleavage sequences, e.g., a furin cleavage sequence to remove the 2A peptide residues attached to the heavy chain or light chain. Exemplary 2A peptides are described, e.g., in Chng et al MAbs 7: 403-412, 20115, and Lin et al. Front. Plant Sci. 9:1379, 2018, the disclosures of which are hereby incorporated by reference in their entirety.

In some embodiments, the antibody is a single-chain antibody or antigen-binding fragment thereof expressed from a single transgene.

nORF Replacement

The present invention also features methods of treating a disease by administering or providing a WT nORF or a protein encoded by the WT nORF. The therapy may restore the encoded protein product of the WT nORF without the sequence variant, such as to replace the WT nORF that is no longer present due to the mutation. The therapy may include providing the protein product or a polynucleotide encoding the protein product. The method may include providing a vector (e.g., a viral vector) that encodes the protein product. Alternatively, the protein encoded by the nORF may be administered directly, e.g., as an enzyme replacement therapy. The WT nORF or a polynucleotide encoding the WT nORF (e.g., a vector, e.g., a viral vector) may be formulated, e.g., in a pharmaceutical composition containing a pharmaceutically acceptable carrier. The composition can be administered by any suitable method known in the art to the skilled artisan. The composition may be formulated in a virus or a virus-Ike particle.

In some embodiments, the length of the WT nORF is less than about 100 amino acids (e.g., from about 50 to 100, 50 to 90.50 to 80, 60 to 90, 60 to 80, 70 to 100, 70 to 90, 70 to 80, 80 to 100, or 90 to 100 amino acids).

### Viral Vectors for Expression

Viral genomes provide a rich source of vectors that can be used for the efficient delivery of exogenous genes into a mammalian cell. The gene to be delivered may include an inhibitor that targets a variant nORF, such as an RNA (e.g., an aptamer, a miRNA, an antisense RNA, an shRNA, or an siRNA). Alternatively, the gene to be delivered may include the WT nORF for replacement. Viral genomes are particularly useful vectors for gene delivery as the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors are a retrovirus (e.g., Retroviridae family viral vector), adenovirus (e.g., Ad5, Ad26, Ad34, Ad35, and Ad48), parvovirus (e.g., an adeno-associated viral (AAV) vector), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses are: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology, Third Edition (Lippincott-Raven, Philadelphia, (1996))). Other examples are murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus. Gibbon ape leukemia virus. Mason Pfizer monkey virus, simian immunodeficiency virus, simian sarcoma virus, Rous sarcoma virus and lentiviruses. Other examples of vectors are described, for example, in McVey et al., (U.S. Pat. No. 5,801,030), the teachings of which are incorporated herein by reference.

### Retroviral Vectors

The delivery vector used in the methods described herein may be a retroviral vector. One type of retroviral vector that may be used in the methods and compositions described herein is a lentiviral vector. Lentiviral vectors (LVs), a subset of retroviruses, transduce a wide range of dividing and non-dividing cell types with high efficiency, conferring stable, long-term expression of the transgene encoding the polypeptide or RNA. An overview of optimization strategies for packaging and transducing LVs is provided in Delenda, The Journal of Gene Medicine 6: S125 (2004), the disclosure of which is incorporated herein by reference.

The use of lentivirus-based gene transfer techniques relies on the in vitro production of recombinant lentiviral particles carrying a highly deleted viral genome in which the agent of interest is accommodated. In particular, the recombinant lentivirus are recovered through the in trans coexpression in a permissive cell line of (1) the packaging constructs, i.e., a vector expressing the Gag-Pol precursors together with Rev (alternatively expressed in trans); (2) a vector expressing an envelope receptor, generally of an heterologous nature; and (3) the transfer vector, consisting in the viral cDNA deprived of all open reading frames, but maintaining the sequences required for replication, encapsidation, and expression, in which the sequences to be expressed are inserted.

A LV used in the methods and compositions described herein may include one or more of a 5′-Long terminal repeat (LTR), HIV signal sequence, HIV Psi signal 5-splice site (SD), delta-GAG element, Rev Responsive Element (RRE), 3′-splice site (SA), elongation factor (EF) 1-alpha promoter and 3′-self inactivating LTR (SIN-LTR). The lentiviral vector optionally includes a central polypurine tract (cPPT) and a woodchuck hepatitis virus post-transcriptional regulatory element (WPRE), as described in U.S. Pat. No. 6,136,597, the disclosure of which is incorporated herein by reference as it pertains to WPRE. The lentiviral vector may further include a pHR′ backbone, which may include for example as provided below.

The Lentigen LV described in Lu et al., Journal of Gene Medicine 6:963 (2004) may be used to express the DNA molecules and/or transduce cells. A LV used in the methods and compositions described herein may a 5-Long terminal repeat (LTR), HIV signal sequence, HIV Psi signal 5′-splice site (SD), delta-GAG element, Rev Responsive Element (RRE), 3′-splice site (SA), elongation factor (EF) 1-alpha promoter and 3′-self inactivating L TR (SIN-LTR). It will be readily apparent to one skilled in the art that optionally one or more of these regions is substituted with another region performing a similar function.

Enhancer elements can be used to increase expression of modified DNA molecules or increase the lentiviral integration efficiency. The LV used in the methods and compositions described herein may include a nef sequence. The LV used in the methods and compositions described herein may include a cPPT sequence which enhances vector integration. The cPPT acts as a second origin of the (+)-strand DNA synthesis and introduces a partial strand overlap in the middle of its native HIV genome. The introduction of the cPPT sequence in the transfer vector backbone strongly increased the nuclear transport and the total amount of genome integrated into the DNA of target cells. The LV used in the methods and compositions described herein may include a Woodchuck Posttranscriptional Regulatory Element (WPRE). The WPRE acts at the transcriptional level, by promoting nuclear export of transcripts and/or by increasing the efficiency of polyadenylation of the nascent transcript, thus increasing the total amount of mRNA in the cells. The addition of the WPRE to LV results in a substantial improvement in the level of expression from several different promoters, both in vitro and in vivo. The LV used in the methods and compositions described herein may include both a cPPT sequence and WPRE sequence. The vector may also include an IRES sequence that permits the expression of multiple polypeptides from a single promoter.

In addition to IRES sequences, other elements which permit expression of multiple polypeptides are useful. The vector used in the methods and compositions described herein may include multiple promoters that permit expression more than one polypeptide. The vector used in the methods and compositions described herein may include a protein cleavage site that allows expression of more than one polypeptide. Examples of protein cleavage sites that allow expression of more than one polypeptide are described in Klump et al., Gene Ther.; 8:811 (2001), Osbom et al., Molecular Therapy 12:569 (2005), Szymczak and Vignali, Expert Opin Biol Ther. 5:627 (2005), and Szymczak et al., Nat Biotechnol. 22:589 (2004), the disclosures of which are incorporated herein by reference as they pertain to protein cleavage sites that allow expression of more than one polypeptide. It will be readily apparent to one skilled in the art that other elements that permit expression of multiple polypeptides identified in the future are useful and may be utilized in the vectors suitable for use with the compositions and methods described herein.

The vector used in the methods and compositions described herein may, be a clinical grade vector.

The viral vectors (e.g., retroviral vectors, e.g., lentiviral vectors) may include a promoter operably coupled to the transgene encoding the polypeptide or the polynucleotide encoding the RNA to control expression. The promoter may be, e.g., a ubiquitous promoter. Alternatively, the promoter may be, e.g., a tissue specific promoter, such as a myeloid cell-specific or hepatocyte-specific promoter. Suitable promoters that may be used with the compositions described herein include CD11b promoter, sp146/p47 promoter, CD68 promoter, sp146/gp9 promoter, elongation factor 1α (EF1α) promoter, EF1α short form (EFS) promoter, phosphoglycerate kinase (PGK) promoter, α-globin promoter, and β-globin promoter. Other promoters that may be used include, e.g., DC172 promoter, human serum albumin promoter, alphal antitrypsin promoter, thyroxine binding globulin promoter. The DC172 promoter is described in Jacob, et al. Gene Ther. 15:594-603, 2008, hereby incorporated by reference in its entirety.

The viral vectors (e.g., retroviral vectors, e.g., lentiviral vectors) may include an enhancer operably coupled to the transgene encoding the polypeptide or the polynucleotide encoding the RNA to control expression. The enhancer may include a β-globin locus control region (OLCR).

Methods of Measuring nORF Gene Expression

Preferably, the compositions and methods of the disclosure are used to facilitate expression of a WT nORF at physiologically normal levels in a patient (e.g., a human patient). The therapeutic agents of the disclosure, for example, may reduce the variant nORF expression in a human subject. For example, the therapeutic agents of the disclosure may reduce variant nORF expression e.g., by about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, 98%, or 99%.

The expression level of the nORF expressed in a patient can be ascertained, for example, by evaluating the concentration or relative abundance of mRNA transcripts derived from transcription of the nORF. Additionally, or alternatively, expression can be determined by evaluating the concentration or relative abundance of the nORF following transcription and/or translation of an inhibitor that decreases an amount of the variant nORF. Protein concentrations can also be assessed using functional assays, such as MDP detection assays. Expression can be evaluated by a number of methodologies known in the art, Including, but not limited to, nucleic acid sequencing, microarray analysis, proteomics, in-situ hybridization (e.g., fluorescence in-situ hybridization (FISH)), amplification-based assays, in situ hybridization, fluorescence activated cell sorting (FACS), northern analysis and/or PCR analysis of mRNAs.

### Nucleic Acid Detection

Nucleic acid-based methods for determining expression (e.g., of an RNA inhibitor or an RNA encoding the WT nORF) detection that may be used in conjunction with the compositions and methods described herein include imaging-based techniques (e.g., Northern blotting or Southern blotting). Such techniques may be performed using cells obtained from a patient following administration of the polynucleotide encoding the agent. Northern blot analysis is a conventional technique well known in the art and is described, for example, in Molecular Cloning, a Laboratory Manual, second edition, 1989, Sambrook, Fritch, Maniatis, Cold Spring Harbor Press, 10 Skyline Drive, Plainview, NY 11803-2500. Typical protocols for evaluating the status of genes and gene products are found, for example in Ausubel et al., eds., 1995, Current Protocols in Molecular Biology, Units 2 (Northern Blotting), 4 (Southern Blotting), 15 (Immunoblotting) and 18 (PCR Analysis).

Detection techniques that may be used in conjunction with the compositions and methods described herein to evaluate nORF expression further include microarray sequencing experiments (e.g., Sanger sequencing and next-generation sequencing methods, also known as high-throughput sequencing or deep sequencing). Exemplary next generation sequencing technologies include, without limitation, Illumina sequencing, Ion Torrent sequencing, 454 sequencing, SOLiD sequencing, and nanopore sequencing platforms. Additional methods of sequencing known in the art can also be used. For instance, expression at the mRNA level may be determined using RNA-Seq (e.g., as described in Mortazavi et al., Nat. Methods 5:621-628 (2008) the disclosure of which is incorporated herein by reference in their entirety). RNA-Seq is a robust technology for monitoring expression by direct sequencing the RNA molecules in a sample. Briefly, this methodology may involve fragmentation of RNA to an average length of 200 nucleotides, conversion to cDNA by random priming, and synthesis of double-stranded cDNA (e.g., using the Just cDNA DoubleStranded cDNA Synthesis Kit from Agilent Technology). Then, the cDNA is converted into a molecular library for sequencing by addition of sequence adapters for each library (e.g., from Illumina®/Solexa), and the resulting 50-100 nucleotide reads are mapped onto the genome.

Expression levels of the nORF may be determined using microarray-based platforms (e.g., single-nucleotide polymorphism arrays), as microarray technology offers high resolution. Details of various microarray methods can be found in the literature. See, for example, U.S. Pat. No. 6,232,068 and Pollack et al., Nat. Genet. 23:41-46 (1999), the disclosures of each of which are incorporated herein by reference in their entirety. Using nucleic acid microarrays, mRNA samples are reverse transcribed and labeled to generate cDNA. The probes can then hybridize to one or more complementary nucleic acids arrayed and immobilized on a solid support. The array can be configured. for example, such that the sequence and position of each member of the array is known. Hybridization of a labeled probe with a particular array member indicates that the sample from which the probe was derived expresses that gene. Expression level may be quantified according to the amount of signal detected from hybridized probe-sample complexes. A typical microarray experiment involves the following steps: 1) preparation of fluorescently labeled target from RNA isolated from the sample, 2) hybridization of the labeled target to the microarray, 3) washing, staining, and scanning of the array, 4) analysis of the scanned image and 5) generation of gene expression profiles. One example of a microarray processor is the Affymetrix GENECHIP® system, which is commercially available and includes arrays fabricated by direct synthesis of oligonucleotides on a glass surface. Other systems may be used as known to one skilled in the art.

Amplification-based assays also can be used to measure the expression level of the nORF or RNA in a target cell following delivery to a patient. In such assays, the nucleic acid sequences of the gene act as a template in an amplification reaction (for example, PCR, such as qPCR). In a quantitative amplification, the amount of amplification product is proportional to the amount of template in the original sample. Comparison to appropriate controls provides a measure of the expression level of the gene, corresponding to the specific probe used, according to the principles described herein. Methods of real-time qPCR using TaqMan probes are well known in the art. Detailed protocols for real-time qPCR are provided, for example, in Gibson et al., Genome Res. 6:995-1001 (1996), and in Heid et al., Genome Res. 6:986-994 (1996), the disclosures of each of which are incorporated herein by reference in their entirety. Levels of gene expression as described herein can be determined by RT-PCR technology. Probes used for PCR may be labeled with a detectable marker, such as, for example, a radioisotope, fluorescent compound, bioluminescent compound, a chemiluminescent compound, metal chelator, or enzyme.

### Protein Detection

Expression of the nORF can additionally be determined by measuring the concentration or relative abundance of a corresponding protein product (e.g., the WT nORF or the variant nORF). Protein levels can be assessed using standard detection techniques known in the art. Protein expression assays suitable for use with the compositions and methods described herein include proteomics approaches, Immunohistochemical and/or western blot analysis, Immunoprecipitation, molecular binding assays, ELISA, enzyme-linked immunofiltration assay (ELIFA), mass spectrometry, mass spectrometric immunoassay, and biochemical enzymatic activity assays. In particular, proteomics methods can be used to generate large-scale protein expression datasets in multiplex. Proteomics methods may utilize mass spectrometry to detect and quantify polypeptides (e.g., proteins) and/or peptide microarrays utilizing capture reagents (e.g., antibodies) specific to a panel of target proteins to identify and measure expression levels of proteins expressed in a sample (e.g., a single cell sample or a multi-cell population).

Exemplary peptide microarrays have a substrate-bound plurality of polypeptides, the binding of an oligonucleotide, a peptide, or a protein to each of the plurality of bound polypeptides being separately detectable. Alternatively, the peptide microarray may include a plurality of binders, including, but not limited to, monoclonal antibodies, polyclonal antibodies, phage display binders, yeast two-hybrid binders, aptamers, which can specifically detect the binding of specific oligonucleotides, peptides, or proteins. Examples of peptide arrays may be found in U.S. Pat. Nos. 6,268,210, 5,766,960, and 5,143,854, the disclosures of each of which are incorporated herein by reference in their entirety.

Mass spectrometry (MS) may be used in conjunction with the methods described herein to identify and characterize expression of the nORF in a cell from a patient (e.g., a human patient) following delivery of the transgene encoding the nORF. Any method of MS known in the art may be used to determine, detect, and/or measure a protein or peptide fragment of interest, e.g., LC-MS, ESI-MS, ESI-MS/MS, MALDI-TOF-MS, MALDI-TOF/TOF-MS, tandem MS, and the like. Mass spectrometers generally contain an ion source and optics, mass analyzer, and data processing electronics. Mass analyzers include scanning and ion-beam mass spectrometers, such as time-of-flight (TOF) and quadruple (Q), and trapping mass spectrometers, such as Ion trap (IT), Orbitrap, and Fourier transform ion cyclotron resonance (FT-ICR), may be used in the methods described herein. Details of various MS methods can be found in the literature. See, for example, Yates et al., Annu. Rev. Biomed. Eng. 11:49-79, 2009, the disclosure of which is incorporated herein by reference in its entirety.

Prior to MS analysis, proteins in a sample obtained from the patient can be first digested into smaller peptides by chemical (e.g., via cyanogen bromide cleavage) or enzymatic (e.g., trypsin) digestion. Complex peptide samples also benefit from the use of front-end separation techniques, e.g., 2D-PAGE, HPLC, RPLC, and affinity chromatography. The digested, and optionally separated, sample is then ionized using an ion source to create charged molecules for further analysis. Ionization of the sample may be performed, e.g., by electrospray ionization (ESI), atmospheric pressure chemical ionization (APCI), photolonization, electron ionization, fast atom bombardment (FAB)/liquid secondary ionization (LSIMS), matrix assisted laser desorption/ionization (MALDI), field ionization, field desorption, thermospray/plasmaspray ionization, and particle beam ionization. Additional information relating to the choice of ionization method is known to those of skill in the art.

After ionization, digested peptides may then be fragmented to generate signature MS/MS spectra. Tandem MS, also known as MS/MS, may be particularly useful for analyzing complex mixtures. Tandem MS involves multiple steps of MS selection, with some form of ion fragmentation occurring in between the stages, which may be accomplished with individual mass spectrometer elements separated in space or using a single mass spectrometer with the MS steps separated in time. In spatially separated tandem MS, the elements are physically separated and distinct, with a physical connection between the elements to maintain high vacuum. In temporally separated tandem MS, separation is accomplished with ions trapped in the same place, with multiple separation steps taking place over time. Signature MS/MS spectra may then be compared against a peptide sequence database (e.g., SEQUEST). Post-translational modifications to peptides may also be determined, for example, by searching spectra against a database while allowing for specific peptide modifications.

### Diseases

A number of diseases are known in the art that are associated with a variant. However, the present invention contemplates treatment of a disease in which the variant may be benign in the associated canonical ORF of the gene but has a deleterious effect in the nORF. The skilled artisan practicing the invention can identify the variant in the nORF using the methods described herein. Alternatively, the skilled artisan could identify a benign variant in a cORF and determined whether that cORF contains an associated nORF. The skilled person may further determine whether the variant is present within the nORF and whether this variant introduces a stop codon or the loss of a stop codon.

In some embodiments, the disease is cancer (e.g., breast cancer or Medullary thyroid carcinoma). The gene may be BRCA2. The gene may be RET. In some embodiments, the gene is selected from the group consisting of TTN, TP53, EGFR, FAT1, MACF1, TSC2, NOTCH1, ANK2, MYC, NEB, NLRP2, CREBBP, ANAPC5, DST, EXT1, NF1, AR1D1A, ATM, CTNNA2, and JAK1.

When treating cancer, the method may reduce the size (e.g., by 10%, 20%, 30%, 40%, 50%, 60%, 70%, 80%, 90%, 95%, or 99%) of a tumor (e.g., a breast tumor).

In some embodiments (a) the disease is Leber congenital amaurosis, and the gene is NMNAT1; (b) the disease is Charcot Marie Tooth disease type 1B, and the gene is MPZ; (c) the disease is Spastic paraplegia autosomal dominant, and the gene is SPAST; (d) the disease is Pulmonary arterial hypertension, and the gene is BMPR2; (e) the disease is Coproporphyria, and the gene is CPOX; (f) the disease is Epileptic encephalopathy early onset, and the gene is ALDH7A1; (g) the disease is Alpha-AASA dehydrogenase deficiency, and the gene is ALDH7A1; (h) the disease is Mucopolysaccharidosis VII, and the gene is GUSB; (i) the disease is Cowden disease, and the gene is PTEN; (J) the disease is Beta thalassaemla, and the gene is HBB; (k) the disease is Multiple endocrine neoplasia 1, and the gene is MEN1; (l) the disease is Cerebellar ataxia recurrent liver failure peripheral neuropathy and short stature, and the gene is SCYL1; (m) the disease is Pituitary adenoma, and the gene is AIP; (n) the disease is Marfan syndrome, and the gene is FBN1; (o) the disease is Gangliosidosis GM2, and the gene is HEXA; (p) the disease is Leigh syndrome, and the gene is MRPS34; (q) the disease is Apparent mineralocorticoid excess, and the gene is HSD11B2; (r) the disease is Neurofibromatosis 1, and the gene is NF1; (s) the disease is Osteogenesis imperfecta 1, and the gene is COL1A1; (t) the disease is Hypercholesterolaemia, and the gene is LDLR; (u) the disease is Aicardi-Goutlères syndrome, and the gene is RNASEH2A; (v) the disease is Hyperferritinaemia cataract syndrome, and the gene is FTL; (w) the disease is Retinitis pigmentosa, and the gene is PRPF31; (x) the disease is Neurofibromatosis 2, and the gene is NF2; (y) the disease is Pyridoxine-dependent epilepsy, and the gene is ALDH7A1; (z) the disease is Hypotrichosis 4, and the gene is HR; (aa) the disease is Somatotroph adenoma, and the gene is AIP; (bb) the disease is Gm2 gangliosidosis, subacute, and the gene is HEXA; (cc) the disease is Combined oxidative phosphorylation deficiency 32, and the gene is MRPS34; or (dd) the disease is Aicardi Goutieres syndrome 4, and the gene is MRPS34.

In some embodiments, the disease and the gene are selected from Table 3.

In some embodiments, the disease and the gene are selected from Table 4.

In some embodiments, the disease and the gene are selected from Table 5.

In some embodiments, the disease is selected from the list consisting of amyotrophic lateral sclerosis, martan syndrome, myasthenic syndrome, congenital, Charcot-Marie-Tooth disease, neural tube defects, Ehlers-Danlos syndrome, cortical cataract, dyssegmental dysplasia, Diamond-Blackfan anemia, familial hypercholesterolemia, reticular dysgenesis, dystonia, severe congenital neutropenia, hyperinsulinism, noonan syndrome, mitochondrial cytopathy, Melnick-Needles syndrome, frontometaphyseal dysplasia, spastic paraplegia, Baraitser-Winter syndrome, peripheral axonal neuropathy, mucopolysaccharidosis, lissencephaly 2, maple syrup urine disease, myofibrillar myopathy, Pitt-Hopkins-like syndrome 1, weaver syndrome, arrhythmia, cardiomyopathy, glycogen storage disease of heart, neuronal ceroid lipofuscinosis, primary autosomal recessive microcephaly 1, Werner syndrome, Spherocytosis, Waardenburg syndrome, ciliary dyskinesia, epidermolysis bullosa simplex, Brown-Vialetto-Van Laere syndrome, amyotrophic lateral sclerosis, hyperphosphatasia with mental retardation syndrome, distal arthrogryposis, choreoacanthocytosis, phosphoserine aminotransferase deficiency, spinal muscular atrophy, congenital cataract, thoracic aortic aneurysm and aortic dissection, familial dysautonomia, Bardet-Biedl syndrome, amyloidosis, early infantile epileptic encephalopathy, Osler hemorrhagic telangiectasia syndrome, coenzyme Q10 deficiency, Walker-Warburg congenital muscular dystrophy, spinocerebellar ataxia autosomal recessive, Leigh syndrome, Ehlers-Dankos syndrome, Adams-Oliver syndrome, congenital generalized lipodystrophy, Barakat syndrome, primary open angle glaucoma, Warburg micro syndrome, long QT syndrome, multiple endocrine neoplasia, pol III-related leukodystrophy, moyamoya disease|, dilated cardiomyopathy, cutis laxa-corneal clouding-oligophrenla syndrome, Infantile spasms, Hermansky-Pudlak syndrome, Medulloblastoma, myofibrillar myopathy, Costello syndrome, seizure, neuronal ceroid lipofuscinosis, Beckwith-Wiedemann syndrome, Stormorken syndrome, neuronal ceroid lipofuscinosis, Sveinsson chorioretinal atrophy, Wilms tumor, peroxisome biogenesis disorder, syndactyly Cenani Lenz type, xeroderma pigmentosum, hereditary paraganglioma-pheochromocytoma syndromes, multiple endocrine neoplasia, type 1, autosomal recessive cutis laxa type 1, osteopetrosis autosomal recessive 1, osteogenesis imperfecta, recessive, Papillon-Lefevre syndrome, ataxia-telangiectasia syndrome, myofibrillar myopathy, 6-pyruvoyl-tetrahydropterin synthase deficiency, glycogen storage disease, type I, glucose-6-phosphate transport defect, pseudohypoaldosteronism type 2C, pseudohypoaldosteronism type 1, epidermolysis bullosa simplex, keratosis follicularis, Troyer syndrome, neuronal ceroid lipofuscinosis, nemaline myopathy 7, elliptocytosis, methylmalonate semialdehyde dehydrogenase deficiency, ventricular tachycardia, catecholaminergic polymorphic, herpes simplex encephalitis, mosaic variegated aneuploldy, arginine:glycine amidinotransferase deficiency, marfan syndrome, ectopia lentis, Griscelli syndrome type 2, fanconi anemia, progressive sclerosing poliodystrophy, Bloom syndrome, Weill-Marchesani-like syndrome, bare lymphocyte syndrome 2, EEM syndrome, Li-Fraumeni syndrome, Meier-Gorlin syndrome, naxos disease, osteogenesis imperfecta, carney complex, type 1, Howel-Evans syndrome, Majeed syndrome, Niemann-Plck disease, type C, Peutz-Jeghers syndrome, lipodystrophy, partial, acquired, leprechaunism syndrome, rhabdoid tumor predisposition syndrome 2, Aicardi Goutieres syndrome 4, retinitis pigmentosa, recessive, alagille syndrome 1, dyskeratosis congenita, pseudoinflammatory fundus dystrophy, adenylosuccinate lyase deficiency, duchenne muscular dystrophy, Wilson-Turner X-linked mental retardation syndrome, Melnick-Needles syndrome, transcobalamin II deficiency, nephronophthisis-like nephropathy, and Borjeson-Forssman-Lehmann syndrome.

### EXAMPLES

The following examples further illustrate the invention but should not be construed as in any way limiting its scope.

### Example 1

### Results

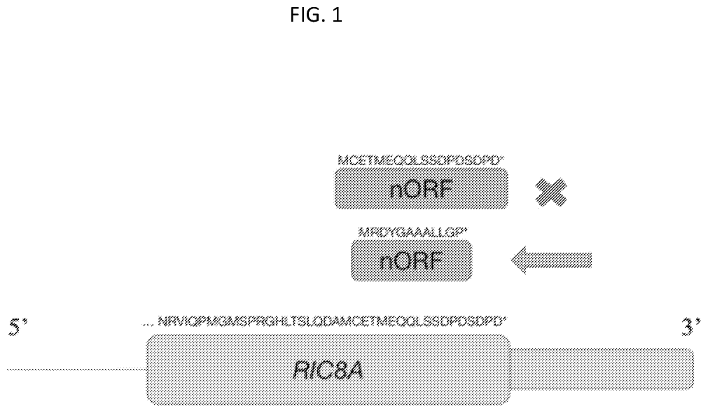

The nORFs dataset contains 194,407 ORFs curated from OpenProt and sORFs.org from canonical proteins (FIG. 1).

We compared this nORF dataset with previously published uORF dataset (McGillivray et al. Nucleic Acids Res 46: 3326-3338, 2018). We note that the sources of uORF entries from McGillivray et al. are from ribosome profiling experiments also used as input for the sORFs.org dataset. Comparing the 188,802 “likely active” uORFs from McGillivray et al. 2018, with the 194,407 nORFs from this work, we find that there are 15,082 entries that are identical or highly similar (share stop codon but differ in start codon) between datasets. The majority of these shared entries fall, as expected, under the nORFs classified as 5′UTR (7,333) or 5′UTR-aftCDS (3,681). The entries in the nORFs dataset not found in the uORF dataset can be attributed to the broader set of experiments used as input from sORFs.org and OpenProt, and the broader focus of any all unannotated ORFs, compared to the specific uORF focus of McGillivray et al. 2018. As the 188,802 “likely active” uORFs from McGillivray et al. 2018 would have been found in sORFs.org dataset, those not found in the nORFs dataset would have been filtered out at one of data curation steps performed (e.g., good/extreme ORF score, longest ORF if similar, removing in-frame entries).

Disease Mutations in nORF Contexts

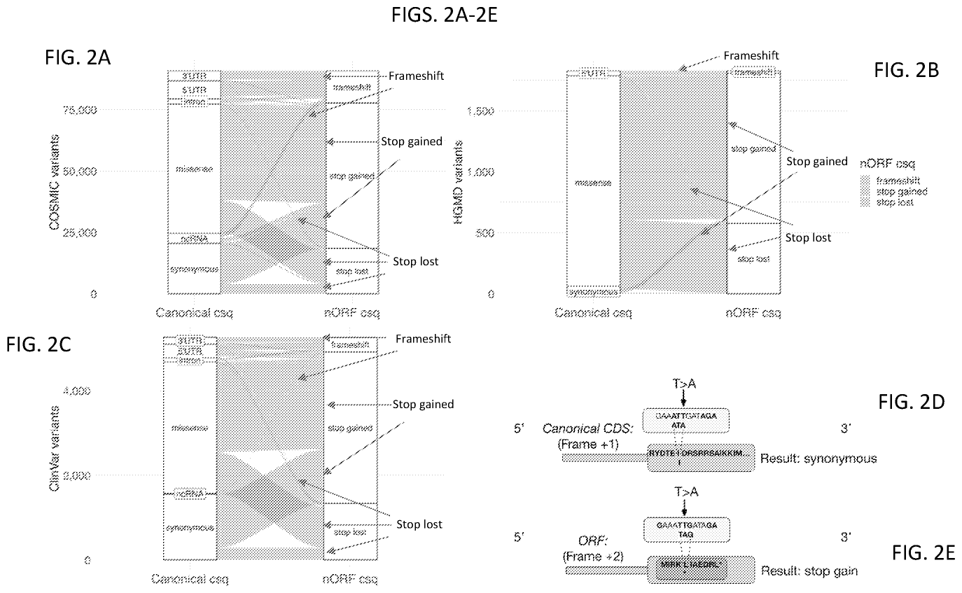

Considering that stop lost and stop gained variants in nORFs show signals of negative selection. we investigated potential disease-causing variants that could be due to these mutation types. We first examined somatic cancer mutations from the Catalogue Of Somatic Mutations In Cancer (COSMIC) database. We annotated the 6.2 million coding and 19.7 million non-coding somatic variants using VEP in the context of nORFs and then canonical annotations. Although COSMIC variant sets are expected to be dominated by passenger mutations, their functional interpretation is key to identifying the cancer-causing genes and variants. We highlight 109K potential frameshift, stop gained, or stop lost variants in nORFs that have a less severe consequence in canonical genes (FIG. 2A; Table 1).

TABLE 1
Number of COSMIC variants with deleterious consequences
in nORFs but benign or missense consequences in canonical frames

| Canonical Csq | nORF csq | Variant Count |
|---|---|---|
| 3′UTR | frameshift | 3694 |
| 3′UTR | stop gained | 369 |
| 3′UTR | stop lost | 147 |
| 5′UTR | frameshift | 6194 |
| 5′UTR | stop gained | 814 |
| 5′UTR | stop lost | 272 |
| intergenic | frameshift | 22 |
| intergenic | stop gained | 26 |
| intergenic | stop lost | 12 |
| intron | frameshift | 1426 |
| intron | stop gained | 308 |
| intron | stop lost | 336 |
| missense | stop gained | 49380 |
| missense | stop lost | 16347 |
| ncRNA | frameshift | 2004 |
| ncRNA | stop gained | 1802 |
| ncRNA | stop lost | 517 |
| synonymous | stop gained | 20721 |
| synonymous | stop lost | 4898 |

We then performed a similar analysis to annotate known human disease variants present in the Human Gene Mutation Database (HGMD) and ClinVar databases. We identified 1,852 variants from HGMD and 5,269 variants from ClinVar that are frameshift, stop gained, or stop lost variants in nORFs, but have less severe consequences in canonical genes (FIGS. 2B and 2C; Table 2).

TABLE 2
Number of HGMD and Clinvar mutations with deleterious consequences
in nORFs but benign or missense consequences in canonical frames.

| Canonical Csq | nORF csq | Variant Count |
|---|---|---|
| 3′UTR | frameshift | 123 |
| 3′UTR | stop gained | 37 |
| 3′UTR | stop lost | 14 |
| 5′UTR | frameshift | 178 |
| 5′UTR | stop gained | 144 |
| 5′UTR | stop lost | 44 |
| intron | frameshift | 64 |
| intron | stop gained | 14 |
| intron | stop lost | 25 |
| missense | stop gained | 3316 |
| missense | stop lost | 1541 |
| ncRNA | frameshift | 13 |
| ncRNA | stop gained | 12 |
| ncRNA | stop lost | 3 |
| synonymous | stop gained | 1306 |
| synonymous | stop lost | 287 |

To create a short list of disease mutations most likely to have a nORF related cause, we further prioritized the COSMIC, HGMD and ClinVar disease mutations. Specifically, we identified top cancer-associated genes with mutations with benign consequences in CDS but with deleterious consequences in the nORFs (Table 3. Cancer genes with benign COSMIC mutation consequences in CDS but with deleterious consequences in the nORF), 34 HGMD variants classified as disease causing (Table 4) and 14 ClinVar variants classified as pathogenic or likely pathogenic (Table 5. ClinVar pathogenic mutations potentially explained by nORF consequences) that have benign consequences in canonical annotations but stop loss or stop gain consequences in nORFs. We show an example where a theoretical synonymous disease variant has a stop gained effect on a nORF overlapping canonical CDS (FIGS. 2D and 2E) which would normally be missed as a potential mechanism of pathogenicity.

TABLE 4
HGMD disease mutations potentially explained by nORF consequences

| Chr | POS | ID | CLASS | PHEN | GENE | nORF_csq | vep_csq |
|---|---|---|---|---|---|---|---|
| 1 | 75761337 | CS138290 | FP | Improved_splicing_efficiency | ACADM | stop | synonymous |
| | | | | | | lost | |
| 1 | 85276329 | CM062460 | DP | Advanced_germ_cell_tumors_ | BCL10 | stop | synonymous |
| | | | | association_with | | gained | |
| 1 | 161306880 | CS093741 | DM | Charcot-Marie- | MPZ | stop | synonymous |
| | | | | Tooth_disease_type_1B | | gained | |
| 2 | 38071274 | CM1517003 | DM? | Glaucoma_primary_congenital | CYP1B1 | stop | synonymous |
| | | | | | | gained | |
| 2 | 135126217 | CM1821650 | DM? | Keratoconus | RAB3GAP1 | stop | synonymous |
| | | | | | | gained | |
| 2 | 202519034 | CS1724033 | DM | Pulmonary_arterial_hypertension | BMPR2 | stop | synonymous |
| | | | | | | lost | |
| 5 | 126568296 | CM188397 | DM | Epileptic_encephalopathy_ | ALDH7A1 | stop | synonymous |
| | | | | early_onset | | gained | |
| 5 | 126568296 | CS075064 | DM | Alpha- | ALDH7A1 | stop | synonymous |
| | | | | AASA_dehydrogenase_deficiency | | gained | |
| 6 | 108074544 | CS136721 | DM? | Osteopetrosis_craniosynostosis_ | OSTM1 | stop | synonymous |
| | | | | and_Chiari_malformation_type_I | | gained | |
| 7 | 65967767 | CS951430 | DM | Mucopolysaccharidosis_VII | GUSB | stop | synonymous |
| | | | | | | gained | |
| 7 | 105536605 | CS147935 | DM? | Breast_cancer | RINT1 | stop | synonymous |
| | | | | | | gained | |
| 8 | 89982788 | CM181674 | DM? | Breast_cancer | NBN | stop | synonymous |
| | | | | | | gained | |
| 9 | 134811374 | CM1821670 | DM? | Keratoconus | COL5A1 | stop | synonymous |
| | | | | | | gained | |
| 10 | 43114490 | CS1815922 | DM | Medullary_thyroid_carcinoma_ | RET | stop | synonymous |
| | | | | modifier_of | | gained | |
| 11 | 64807945 | CM193341 | DM | Multiple_endocrine_neoplasia_1 | MEN1 | stop | synonymous |
| | | | | | | gained | |
| 11 | 65526207 | CS191427 | DM | Cerebellar_ataxia_recurrent_ | SCYL1 | stop | synonymous |
| | | | | liver_failure_peripheral_ | | gained | |
| | | | | neuropathy_and_short_stature | | | |
| 11 | 67487155 | CS104882 | DM | Pituitary_adenoma | AIP | stop | synonymous |
| | | | | | | gained | |
| 11 | 67490807 | CM1719875 | DM | Pituitary_adenoma | AIP | stop | synonymous |
| | | | | | | gained | |
| 11 | 67490807 | CS083216 | DM | Pituitary_adenoma | AIP | stop | synonymous |
| | | | | | | gained | |
| 11 | 108235776 | CM1823080 | DM? | Breast_cancer_female | ATM | stop | synonymous |
| | | | | | | gained | |
| 11 | 108244887 | CM1822751 | DM? | Breast_cancer_male | ATM | stop | synonymous |
| | | | | | | lost | |
| 11 | 108302909 | CM1823107 | DM? | Breast_cancer_female | ATM | stop | synonymous |
| | | | | | | lost | |
| 12 | 12131981 | CM073186 | DP | Alzheimer_disease_late- | LRP6 | stop | synonymous |
| | | | | onset_association_with | | gained | |
| 13 | 20189144 | CM1718632 | DM? | Hearing_loss_non- | GJB2 | stop | synonymous |
| | | | | syndromic | | gained | |
| 13 | 32363194 | CS118467 | DM | Breast_cancer | BRCA2 | stop | synonymous |
| | | | | | | gained | |
| 15 | 48437004 | CS118012 | DM | Marfan_syndrome | FBN1 | stop | synonymous |
| | | | | | | gained | |
| 15 | 48489954 | CM1812549 | DM? | Thoracic_aortic_disorder | FBN1 | stop | synonymous |
| | | | | | | gained | |
| 15 | 72349093 | CS045964 | DM | Gangliosidosis_GM2 | HEXA | stop | synonymous |
| | | | | | | gained | |
| 16 | 67436355 | CS032075 | DM | Apparent_mineralocorticoid_ | HSD11B2 | stop | synonymous |
| | | | | excess | | gained | |
| 17 | 1661682 | CM1613930 | DM? | Retinal_disease | PRPF8 | stop | synonymous |
| | | | | | | gained | |
| 18 | 21042578 | CM135652 | DP | Tetralogy_of_Fallot_ | ROCK1 | stop | synonymous |
| | | | | predisposition_to | | gained | |
| 19 | 11089596 | CM1618236 | DM | Hypercholesterolaemia | LDLR | stop | synonymous |
| | | | | | | gained | |
| 19 | 11105413 | CM071832 | DM | Hypercholesterolaemia | LDLR | stop | synonymous |
| | | | | | | gained | |
| 19 | 11105527 | CS081932 | DM | Hypercholesterolaemia | LDLR | stop | synonymous |
| | | | | | | gained | |
| 19 | 12806748 | CS137971 | DM | Aicardi- | RNASEH2A | stop | synonymous |
| | | | | Goutieres_syndrome | | gained | |
| 19 | 54122591 | CM1723658 | DM | Retinitis_pigmentosa | PRPF31 | stop | synonymous |
| | | | | | | gained | |
| 22 | 19963684 | CM970391 | DFP | Schizoaffective_disorder_ | COMT | stop | synonymous |
| | | | | association_with | | gained | |
| 22 | 32857305 | CM1816018 | DP | Bicuspid_aortic_valve_and_ | TIMP3 | stop | synonymous |
| | | | | aortopathy_in_Turner_ | | gained | |
| | | | | syndrome_association_with | | | |

### Discussion

Following the advent of proteogenomics, ribosome profiling, and massively parallel sequencing studies, a key observation was that the entire genome has the potential to encode transcriptional and translational products. It was observed that noncanonical transcription and translation is not bound by classical motifs for transcriptional start or stop sites, polyadenylation, AUG start codons, single CDS per transcript, or numerous other signatures associated with the conventional gene definitions. Beyond the lack of conventional signatures to identify them, there is no consensus on how nORFs should be classified, with research groups often focusing on specific types or sizes of nORFs. We have undertaken a systematic analysis to collate and reclassify these nORFs into an accessible dataset available to the wider community. This dataset was created with the goal of facilitating investigations Into nORF signatures for transcription, translation, regulation, and function. In this study, we curated and annotated 194,407 nORFs with translation evidence from MS or ribosome profiling and assessed their functional significance using global genomic properties. We found signals of functional importance for nORFs from negative selection against classes of nORF variants and disease mutations potentially explained by nORFs consequences.

Investigation of this showed that numerous variants in disease mutation databases could have nORF related mechanisms of pathogenicity via stop lost or stop gained mutations. We identified candidate HGMD disease mutations and ClinVar pathogenic/likely-pathogenic mutations with benign effects in canonical genes for which we believe nORF consequences should be considered as possible mechanisms of pathogenicity, similar to uORF perturbing variants known to be disease causing. These examples highlight the potential impact of annotating disease mutations for their nORF consequence.

### Methods

Selection of Sources for Evidence of nORFs

Three existing databases with entries that qualify as nORFs were considered for inclusion in the nORFs dataset: OpenProt, sORFs.org, and SmProt. SmProt was not used due to inconsistencies in data (e.g. incorrect genomic coordinate annotations) and lack of details in their methods to reanalyse the data, specifically in regard to their MS evidence. By contrast, OpenProt and sORFs.org have shown commitment to providing consistent, verifiable, and maintained data, and were therefore used as the main sources for the nORFs dataset.

OpenProt (Release 1.3) predicts all possible ORFs with an ATG start codon and a minimum length of 30 codons that map to an Ensembl or RefSeq transcript. They identified 607,456 alternate ORFs (altORFs) that are neither canonical ORFs, nor an isoform of those ORFs, but in noncoding regions or an alternate frame to canonical CDS. Although OpenProt maps to both Ensembl and RefSeq transcripts, we focus exclusively on the Ensembi annotations for compatibility with the sORFs.org dataset and other downstream analyses. From the altORFs mapped to Ensembl transcripts, we consider the 26,480 aftORFs with translation evidence from MS (21,708). ribosome profiling (5,059), or both (398).

The sORFs.org database (downloaded Apr. 30, 2019) uses notably different inclusion criteria, annotating ‘sORFs’ with translation evidence from 43 human ribosome profiling experiments, then adding MS evidence found in publicly available datasets. The sORFs are defined as ORFs between and 100 codons using any of four start codons: ‘ATG’, ‘CTG’, ‘TTG’, or ‘GTG’, and are not restricted to known transcripts.

Curation of nORFs

The curation steps we performed to create a nORF dataset are detailed in FIG. 1. The final dataset that we created a) contains only nORFs with translation evidence from either MS or ribosome profiling b) contains no duplicate or highly similar entries and c) contains only ORFs clearly distinct from currently annotated canonical proteins.

Next, the OpenProt and sORFs.org datasets were merged, 1,028 redundant entries between the datasets were removed, and 1,976 cases of ambiguous start sites between the two datasets were resolved by again taking the longest ORF, resulting in a merged total of 233,021 entries. The small number of overlapping or similar entries between the two datasets can be partly attributed to different inclusion criteria for ORFs between the databases (i.e. ORF length, start codon, transcript requirement) and the main source of entries (sORFs from ribosome profiling and OpenProt predominantly from MS).

Finally, we separated all entries that were in-frame with canonical CDS, as the translation evidence from these entries cannot be unambiguously resolved as to whether they are from a canonical protein product or an independent nORF embedded within a canonical protein. We identified 38,614 such entries and removed them, leaving a total of 194,407 entries in the final nORFs dataset. An example case is shown in FIG. 1 where two small ORFs overlap the CDS of the RICA gene. One of these ORFs is in the same frame as the RICA CDS and was therefore filtered out, whereas the second ORF is in a different frame and retained in the dataset. Following this final curation step all entries in the nORF dataset that overlap canonical CDS are in a different frame from and do not share amino acid sequence with that CDS.

Annotation of nORFs

We annotated each nORF with reference to human GENCODE (v30) gene. The annotation categories included nORFs mapping to UTRs or CDS of protein coding transcripts, ncRNAs, or intergenic regions. When multiple annotations were possible, due to multiple transcripts in a region, annotations were prioritized by first selecting full overlaps with protein coding transcripts, particularly those that overlap canonical CDS in an alternative reading frame (altCDS), followed by full overlaps with ncRNA transcripts, then by partial transcript overlaps, and finally intronic or intergenic regions.

Using GENCODE 34 (latest version) our pipeline identifies 194,291 rather than 194,407 nORFs, meaning that between releases 30 and 34, 116 nORFs became part of canonical CDS as newly identified genes or as part of new coding transcripts of existing genes. We find it encouraging that some nORFs are becoming canonical CDS and plan to regularly update our GENCODE reference in future iterations of the nORFs database.

### Database and Web Platform

To reduce the threshold of accessibility, databases need to be accessible with minimal requirements of tools or prior knowledge. We therefore built an online platform with Representational State Transfer (REST) application programming interface (API) functionality. This online platform acts as an entry and lookup point for individual entries, while the REST API is feature compatible with existing bioinformatics pipelines. We made the curated and annotated GRCh38 raw dataset available in BED and GTF format as well as a downloadable nORFs.org UCSC track. Considering reproducible research guidelines, we used git as a versioning tool and uploaded the repository to GitHub under an MIT license (github.com/PrabakaranGroup/nORFs.org).

### Variant Annotation

Variant annotation was carried out using version 96 of VEP to investigate the consequences of variants in the context of canonical frames and nORFs. Variant sets were obtained for annotation as VCFs. These included gnomAD genomes and exomes (release 2.1.1), HGMD (pro release 2019.2), ClinVar (release 2019 0708), and COSMIC coding and noncoding mutations (v89). Each set of variants was annotated for their most severe consequence as defined by VEP with respect to a) canonical gene annotations, corresponding to GENCODE 30 in GRCh38 or GENCODE 30 lifted over to GRCh37 and b) nORF annotations provided as a custom GTF in the appropriate genome assembly.

When examining possible disease mutations that could be explained by nORF consequences, we first filtered variants from the disease mutations databases (COSMIC, HGMD, and ClinVar) to remove those with strongly deleterious annotations in canonical proteins (i.e., essential splice, frameshift, stop gained, stop lost, start lost). We then further filtered these variant sets to those with possible pathogenic consequences in nORFs (stop lost, stop gained, and frameshift).

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth and follows in the scope of the claims.

Other embodiments are within the claims.

Lengthy table referenced here
US20240055076A1-20240215-T00001
Please refer to the end of the specification for access instructions.

LENGTHY TABLES
The patent application contains a lengthy table section. A copy of the table is available in electronic form from the USPTO web site (https://seqdata.uspto.gov/?pageRequest=docDetail&DocID=US20240055076A1). An electronic copy of the table will also be available from the USPTO upon request and payment of the fee set forth in 37 CFR 1.19(b)(3).

1. A method of treating a disease in a subject comprising:
(a) identifying a sequence variant in a canonical open reading frame (cORF) of a gene and a disease associated therewith;
(b) identifying a sequence of a novel open reading frame (nORF) of the gene that is distinct from the cORF, wherein the nORF is present in (i) an overlapping region of the cORF in an alternate reading frame, (ii) a 5′ untranslated region (UTR) of the cORF, (iii) a 3′ UTR of the cORF, (Iv) an intronic region of the cORF, or (v) an intergenic region of the cORF, wherein the sequence variant encodes the loss of a stop codon or portion thereof in the nORF, and wherein the absence of the sequence variant does not encode the loss of the stop codon or portion thereof in the nORF; and
(c) administering an inhibitor of the protein encoded by the nORF to the subject treat the disease.

2. A method of treating a disease in a subject comprising administering an inhibitor of a protein encoded by a nORF containing a stop codon to the subject; wherein the subject has previously been identified with:
(a) a sequence variant in a gene comprising a cORF associated with the disease; and
(b) a sequence of the nORF of the gene that is distinct from the cORF, wherein the nORF is present in (i) an overlapping region of the cORF in an alternate reading frame, (ii) a 5′ untranslated region (UTR) of the cORF, (iii) a 3′ UTR of the cORF, (iv) an intronic region of the cORF, or (v) an intergenic region of the cORF, wherein the sequence variant encodes the loss of a stop codon in the nORF, and wherein the absence of the sequence variant does not encode the loss of the stop codon in the nORF.

3. A method of treating a disease in a subject comprising:
(a) identifying a sequence variant in a gene comprising a cORF and a disease associated therewith;
(b) identifying a sequence of a nORF of the gene that is distinct from the cORF, wherein the nORF is present in (i) an overlapping region of the cORF in an alternate reading frame, (ii) a 5′ untranslated region (UTR) of the cORF, (iii) a 3′ UTR of the cORF, (iv) an intronic region of the cORF, or (v) an intergenic region of the cORF, wherein the sequence variant encodes a stop codon or portion thereof in the nORF, and wherein the absence of the sequence variant does not encode the stop codon or portion thereof in the nORF; and
(c) administering an inhibitor of the protein encoded by the nORF to the subject treat the disease.

4. A method of treating a disease in a subject comprising administering an inhibitor of a protein encoded by a nORF to the subject; wherein the subject has previously been identified with:
(a) a sequence variant in a gene comprising a cORF associated with the disease; and
(b) a sequence of the nORF of the gene that is distinct from the cORF, wherein the nORF is present in (i) an overlapping region of the cORF in an alternate reading frame, (ii) a 5′ untranslated region (UTR) of the cORF, (iii) a 3′ UTR of the cORF, (iv) an intronic region of the cORF, or (v) an intergenic region of the cORF, wherein the sequence variant encodes a stop codon in the nORF, and wherein the absence of the sequence variant does not encode the stop codon in the nORF.

5. The method of any one of claims 1 to 4, wherein the inhibitor comprises a small molecule, a polynucleotide, or a polypeptide.

6. The method of claim 5, wherein the polynucleotide comprises a miRNA, an antisense RNA, an shRNA, or an siRNA.

7. The method of claim 5, wherein the polypeptide comprises an antibody or antigen-binding fragment thereof.

8. The method of claim 7, wherein the antigen-binding fragment thereof is an scFv.

9. The method of any one of claims 5 to 8, wherein the inhibitor is encoded by a vector.

10. The method of claim 9, wherein the vector is a viral vector.

11. The method of claim 10, wherein viral vector is selected from the group consisting of a Retroviridae family virus, an adenovirus, a parvovirus, a coronavirus, a rhabdovirus, a paramyxovirus, a picornavirus, an alphavirus, a herpes virus, and a poxvirus.

12. The method of claim 11, wherein the parvovirus viral vector is an adeno-associated virus (AAV) vector.

13. The method of claim 12, wherein the viral vector is a Retroviridae family viral vector.

14. The method of claim 13, wherein the Retroviridae family viral vector is a lentiviral vector.

15. The method of claim 13, wherein the Retroviridae family viral vector is an alpharetroviral vector or a gammaretroviral vector.

16. The method of any one of claims 12 to 15, wherein the Retroviridae family viral vector comprises a central polypurine tract, a woodchuck hepatitis virus post-transcriptional regulatory element, a 5′-LTR, HIV signal sequence, HIV Psi signal 5′-splice site, delta-GAG element, 3′-splice site, and a 3-self inactivating LTR.

17. The method of any one of claims 12 to 16, wherein the viral vector is a pseudotyped viral vector.

18. The method of claim 17, wherein the pseudotyped viral vector is selected from the group consisting of a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.

19. The method of claim 18, wherein the pseudotyped viral vector is a lentiviral vector.

20. The method of any one of claims 17 to 19, wherein the pseudotyped viral vector comprises one or more envelope proteins from a virus selected from vesicular stomatitis virus (VSV), RD114 virus, murine leukemia virus (MLV), feline leukemia virus (FeLV), Venezuelan equine encephalitis virus (VEE), human foamy virus (HFV), walleye dermal sarcoma virus (WDSV), Semliki Forest virus (SFV), Rabies virus, avian leukosis virus (ALV), bovine immunodeficiency virus (BIV), bovine leukemia virus (BLV), Epstein-Barr virus (EBV), Caprine arthritis encephalitis virus (CAEV), Sin Nombre virus (SNV), Cherry Twisted Leaf virus (ChTLV), Simian T-cell leukemia virus (STLV), Mason-Pfizer monkey virus (MPMV), squirrel monkey retrovirus (SMRV), Rous-associated virus (RAV), Fujinami sarcoma virus (FuSV), avian carcinoma virus (MH2), avian encephalomyelitis virus (AEV), Alfa mosaic virus (AMV), avian sarcoma virus CT10, and equine infectious anemia virus (EIAV).

21. The method of claim 20, wherein the pseudotyped viral vector comprises a VSV-G envelope protein.

22. A method of treating a disease in a subject comprising:
(a) identifying a sequence variant in a gene comprising a cORF and a disease associated therewith;
(b) identifying a sequence of a nORF of the gene that is distinct from the cORF, wherein the nORF is present in (i) an overlapping region of the cORF in an alternate reading frame, (ii) a 5′ untranslated region (UTR) of the cORF, (iii) a 3′ UTR of the cORF, (iv) an intronic region of the cORF, or (v) an intergenic region of the cORF, wherein the sequence variant encodes the loss of a stop codon or portion thereof in the nORF, and wherein the absence of the sequence variant does not encode the loss of the stop codon or portion thereof in the nORF; and
(c) providing a protein encoded by the wild-type (WT) nORF containing the stop codon to the subject treat the disease.

23. A method of treating a disease in a subject comprising providing a protein encoded by a WT nORF containing a stop codon to the subject; wherein the subject has previously been identified with:
(a) a sequence variant in a gene comprising a cORF associated with the disease; and
(b) a sequence of the nORF of the gene that is distinct from the cORF, wherein the nORF is present in (i) an overlapping region of the cORF in an alternate reading frame, (ii) a 5′ untranslated region (UTR) of the cORF, (iii) a 3′ UTR of the cORF, (iv) an intronic region of the cORF, or (v) an intergenic region of the cORF, wherein the sequence variant encodes the loss of a stop codon in the nORF, and wherein the absence of the sequence variant does not encode the loss of the stop codon in the nORF.

24. A method of treating a disease in a subject comprising:
(a) identifying a sequence variant in a gene comprising a cORF and a disease associated therewith;
(b) identifying a sequence of a nORF of the gene that is distinct from the cORF, wherein the nORF is present in (i) an overlapping region of the cORF in an alternate reading frame, (ii) a 5′ untranslated region (UTR) of the cORF, (iii) a 3′ UTR of the cORF, (iv) an intronic region of the cORF, or (v) an intergenic region of the cORF, wherein the sequence variant encodes a stop codon or portion thereof in the nORF, and wherein the absence of the sequence variant does not encode the variant stop codon or portion thereof in the nORF; and
(c) providing a protein encoded by the WT nORF without the stop codon to the subject treat the disease.

25. A method of treating a disease in a subject comprising providing a protein encoded by a WT nORF to the subject; wherein the subject has previously been identified with:
(a) a sequence variant in a gene comprising a cORF associated with the disease; and
(b) a sequence of the nORF of the gene that is distinct from the cORF, wherein the nORF is present in (i) an overlapping region of the cORF in an alternate reading frame, (ii) a 5′ untranslated region (UTR) of the cORF, (iii) a 3′ UTR of the cORF, (Iv) an intronic region of the cORF, or (v) an intergenic region of the cORF, wherein the sequence variant encodes the stop codon in the nORF, and wherein the absence of the sequence variant in the WT nORF does not encode the stop codon in the nORF.

26. The method of any one of claims 22 to 25, wherein the method comprises restoring the encoded protein product of the WT nORF without the sequence variant.

27. The method of claim 26, wherein the method comprises providing the protein product or a polynucleotide encoding the protein product.

28. The method of claim 27, wherein the method comprises providing a vector comprising the polynucleotide encoding the protein product.

29. The method of claim 28, wherein the vector is a viral vector.

30. The method of claim 29, wherein viral vector is selected from the group consisting of a Retroviridae family virus, an adenovirus, a parvovirus, a coronavirus, a rhabdovirus, a paramyxovirus, a picornavirus, an alphavirus, a herpes virus, and a poxvirus.

31. The method of claim 30, wherein the parvovirus viral vector is an AAV vector.

32. The method of claim 31, wherein the viral vector is a Retroviridae family viral vector.

33. The method of claim 32, wherein the Retroviridae family viral vector is a lentiviral vector.

34. The method of claim 32, wherein the Retroviridae family viral vector is an alpharetroviral vector or a gammaretroviral vector.

35. The method of any one of claims 29 to 34, wherein the Retroviridae family viral vector comprises a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

36. The method of any one of claims 30 to 35, wherein the viral vector is a pseudotyped viral vector.

37. The method of claim 36, wherein the pseudotyped viral vector is selected from the group consisting of a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.

38. The method of claim 37, wherein the pseudotyped viral vector is a lentiviral vector.

39. The method of any one of claims 35 to 38, wherein the pseudotyped viral vector comprises one or more envelope proteins from a virus selected from vesicular stomatitis virus VSV, RD114 virus, MLV, FeLV, VEE, HFV, WDSV, SFV, Rabies virus, ALV, BIV, BLV, EBV, CAEV, SNV, ChTLV, STLV, MPMV, SMRV, RAV, FuSV, MH2, AEV, AMV, avian sarcoma virus CT10, and EIAV.

40. The method of claim 39, wherein the pseudotyped viral vector comprises a VSV-G envelope protein.

41. The method of any one of claims 1 to 40, wherein the encoded protein product of the nORF is less than about 100 amino acids.

42. The method of any one of claims 1 to 41, further comprising performing a statistical analysis between the variant in the nORF and the disease.

43. The method of claim 42, wherein the statistical analysis measures a positive or negative association between the variant in the nORF and the disease.

44. The method of any one of claims 1 to 43, wherein the disease is cancer.

45. The method of claim 44, wherein the gene is selected from the group consisting of TTN, TP53, EGFR, FAT1, MACF1, TSC2, NOTCH1, ANK2, MYC, NEB, NLRP2, CREBBP, ANAPC5, DST, EXT1, NF1, AR1D1A, ATM, CTNNA2, and JAK1.

46. The method of claim 44, wherein the cancer is breast cancer.

47. The method of claim 46, wherein the gene is BRCA2.

48. The method of claim 44, wherein the cancer is Medullary thyroid carcinoma.

49. The method of claim 48, wherein the gene is RET.

50. The method of any one of claims 1 to 43, wherein:
(a) the disease is Leber congenital amaurosis, and the gene is NMNAT1;
(b) the disease is Charcot Marie Tooth disease type 1B, and the gene is MPZ;
(c) the disease is Spastic paraplegia autosomal dominant, and the gene is SPAST;
(d) the disease is Pulmonary arterial hypertension, and the gene is BMPR2;
(e) the disease is Coproporphyria, and the gene is CPOX;
(f) the disease is Epileptic encephalopathy early onset, and the gene is ALDH7A1;
(g) the disease is Alpha-AASA dehydrogenase deficiency, and the gene is ALDH7A1;
(h) the disease is Mucopolysaccharidosis VII, and the gene is GUSB;
(i) the disease is Cowden disease, and the gene is PTEN;
(j) the disease is Beta thalassaemia, and the gene is HBB;
(k) the disease is Multiple endocrine neoplasia 1, and the gene is MEN1;
(l) the disease is Cerebellar ataxia recurrent liver failure peripheral neuropathy and short stature, and the gene is SCYL1;
(m) the disease is Pituitary adenoma, and the gene is AIP;
(n) the disease is Marfan syndrome, and the gene is FBN1;
(o) the disease is Gangliosidosis GM2, and the gene is HEXA;
(p) the disease is Leigh syndrome, and the gene is MRPS34;
(q) the disease is Apparent mineralocorticoid excess, and the gene is HSD11B2;
(r) the disease is Neurofibromatosis 1, and the gene is NF1;
(s) the disease is Osteogenesis imperfecta I, and the gene is COL1A1;
(t) the disease is Hypercholesterolaemia, and the gene is LDLR;
(u) the disease is Aicardi-Goutières syndrome, and the gene is RNASEH2A;
(v) the disease is Hyperferritinaemla cataract syndrome, and the gene is FTL;
(w) the disease is Retinitis pigmentosa, and the gene is PRPF31;
(x) the disease is Neurofibromatosis 2, and the gene is NF2;
(y) the disease is Pyridoxine-dependent epilepsy, and the gene is ALDH7A1;
(z) the disease is Hypotrichosis 4, and the gene is HR;
(aa) the disease is Somatotroph adenoma, and the gene is AIP;
(bb) the disease is Gm2 gangliosidosis, subacute, and the gene is HEXA;
(cc) the disease is Combined oxidative phosphorylation deficiency 32, and the gene is MRPS34; or
(dd) the disease is Aicardi Goutieres syndrome 4, and the gene is MRPS34.

51. The method of any one of claims 1 to 43, wherein the disease and the gene are selected from Table 3.

52. The method of any one of claims 1 to 43, wherein the disease and the gene are selected from Table 4.

53. The method of any one of claims 1 to 43, wherein the disease and the gene are selected from Table 5.

54. The method of any one of claims 1 to 43, wherein the disease is selected from the list consisting of amyotrophic lateral sclerosis, marfan syndrome, myasthenic syndrome, congenital, Charcot-Marie-Tooth disease, neural tube defects, Ehlers-Danlos syndrome, cortical cataract, dyssegmental dysplasla, Diamond-Blackfan anemia, familial hypercholesterolemia, reticular dysgenesis, dystonia, severe congenital neutropenia, hyperinsulinism, noonan syndrome, mitochondrial cytopathy, Melnick-Needles syndrome, frontometaphyseal dysplasia, spastic paraplegia, Baraltser-Winter syndrome, peripheral axonal neuropathy, mucopolysaccharidosis, lissencephaly 2, maple syrup urine disease, myofibrillar myopathy, Pitt-Hopkins-like syndrome 1, weaver syndrome, arrhythmia, cardiomyopathy, glycogen storage disease of heart, neuronal ceroid lipofuscinosis, primary autosomal recessive microcephaly 1, Werner syndrome, Spherocytosis, Waardenburg syndrome, ciliary dyskinesia, epidermolysis bullosa simplex, Brown-Vialetto-Van Laere syndrome, amyotrophic lateral sclerosis, hyperphosphatasia with mental retardation syndrome, distal arthrogryposis, choreoacanthocytosis, phosphoserine aminotransferase deficiency, spinal muscular atrophy, congenital cataract, thoracic aortic aneurysm and aortic dissection, familial dysautonomia, Bardet-Biedl syndrome, amyloidosis, early infantile epileptic encephalopathy, Osler hemorrhagic telangiectasia syndrome, coenzyme Q10 deficiency, Walker-Warburg congenital muscular dystrophy, spinocerebellar ataxia autosomal recessive, Leigh syndrome, Ehlers-Danlos syndrome, Adams-Oliver syndrome, congenital generalized lipodystrophy, Barakat syndrome, primary open angle glaucoma, Warburg micro syndrome, long QT syndrome, multiple endocrine neoplasia, pol III-related leukodystrophy, moyamoya disease|, dilated cardiomyopathy, cutis laxa-comeal clouding-ollgophrenia syndrome, infantile spasms, Hermansky-Pudlak syndrome, Medulloblastoma, myofibrillar myopathy, Costello syndrome, seizure, neuronal ceroid lipofuscinosis, Beckwith-Wiedemann syndrome, Stormorken syndrome, neuronal ceroid lipofuscinosis, Sveinsson chorioretinal atrophy, Wilms tumor, peroxisome biogenesis disorder, syndactyly Cenani Lenz type, xeroderma pigmentosum, hereditary paraganglioma-pheochromocytoma syndromes, multiple endocrine neoplasia, type 1, autosomal recessive cutis laxa type 1, osteopetrosis autosomal recessive 1, osteogenesis imperfecta, recessive, Papllion-Lefevre syndrome, ataxia-telangiectasia syndrome, myofibrillar myopathy, 6-pyruvoyl-tetrahydropterin synthase deficiency, glycogen storage disease, type I, glucose-6-phosphate transport defect, pseudohypoaldosteronism type 2C, pseudohypoaldosteronism type 1, epidermolysis bullosa simplex, keratosis follicularis, Troyer syndrome, neuronal ceroid lipofuscinosis, nemaline myopathy 7, elliptocytosis, methylmalonate semialdehyde dehydrogenase deficiency, ventricular tachycardia, catecholaminergic polymorphic, herpes simplex encephalitis, mosaic variegated aneuploidy, arginine:glycine amidinotransferase deficiency, marfan syndrome, ectopia lentis, Griscelli syndrome type 2, fanconi anemia, progressive sclerosing poliodystrophy, Bloom syndrome, Weill-Marchesani-like syndrome, bare lymphocyte syndrome 2, EEM syndrome, Li-Fraumeni syndrome, Meler-Gorlin syndrome, naxos disease, osteogenesis imperfecta, carney complex, type 1, Howel-Evans syndrome, Majeed syndrome, Niemann-Plck disease, type C, Peutz-Jeghers syndrome, lipodystrophy, partial, acquired, leprechaunism syndrome, rhabdoid tumor predisposition syndrome 2, Aicardi Goutieres syndrome 4, retinitis pigmentosa, recessive, alagille syndrome 1, dyskeratosis congenita, pseudoinflammatory fundus dystrophy, adenylosuccinate lyase deficiency, duchenne muscular dystrophy, Wilson-Turner X-linked mental retardation syndrome, Melnick-Needles syndrome, transcobalamin II deficiency, nephronophthisis-like nephropathy, and Borjeson-Forssman-Lehmann syndrome.
